(19) United States
(12) Patent Application Publication
(10) Pub. No.: US 2025/0269143 A1
(43) Pub. Date: Aug. 28, 2025

(54) FLEXIBLE VASCULAR CATHETER LINERS MANUFACTURED FROM POLYMER HYBRIDS

(71) Applicant: Veerag Mehta, Plainsboro, NJ (US)

(72) Inventor: Veerag Mehta, Plainsboro, NJ (US)

(21) Appl. No.: 19/055,987

(22) Filed: Feb. 18, 2025

Related U.S. Application Data
(60) Provisional application No. 63/556,459, filed on Feb. 22, 2024.

(51) Int. Cl. A61M 25/00; A61L 29/04; C08L 83/04

(52) U.S. Cl. CPC A61M 25/0045; A61L 29/049; C08L 83/04; A61M 2025/0046; C08L 2203/02; C08L 2207/04

(57) ABSTRACT

A silicone elastomer hybridized with a thermoplastic polymer selected from the group consisting of polyamides; polyolefins; polyurethanes; polyesters; styrenic resins, and blends of any of these thermoplastic polymers and, a vascular catheter liner extruded from said silicone elastomer, hybridized with a thermoplastic polymer.

### CROSS-REFERENCE TO RELATED APPLICATIONS

This application claims benefit of priority to U.S. Provisional Patent application No. 63/556,459 filed on Feb. 22, 2024.

### BACKGROUND OF THE INVENTION

The applicant is not aware of any prior art disclosures.

### BRIEF SUMMARY OF THE INVENTION

Thus, what is disclosed and claimed herein is a silicone elastomer hybridized with a thermoplastic polymer selected from the group consisting of polyamides; polyolefins; polyurethanes; polyesters; styrenic resins, and blends of any of the thermoplastic polymers set forth just above.

In a second embodiment, there is a vascular catheter liner extruded from a silicone elastomer hybridized with a thermoplastic polymer selected from the group consisting of polyamides; polyolefins; polyurethanes; polyesters; styrenic resins, and blends of any of the thermoplastic polymers set forth just above.

### BRIEF DESCRIPTION OF THE SEVERAL VIEWS OF THE DRAWING(S)

FIG. 1 is a prior art extruded vascular catheter showing the various layers of manufacture.

FIG. 2 is an extruded vascular catheter including the catheter liner of this invention showing the various layers of manufacture.

FIG. 3 is a portion of a siloxane molecular chain useful in this invention.

### DETAILED DESCRIPTION OF THE INVENTION

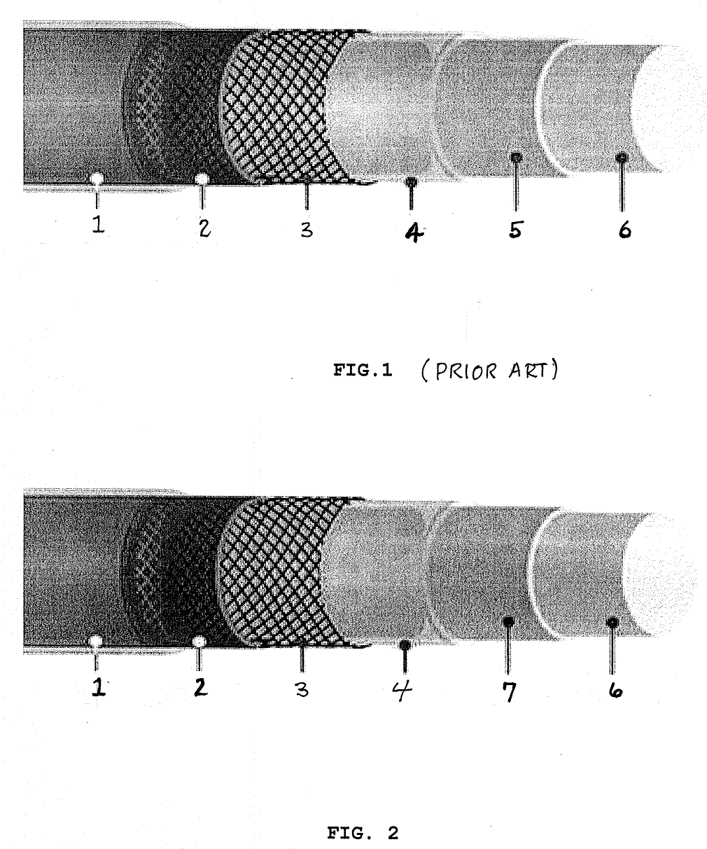

Catheters are often designed using a few layers to provide design and function based on the end application. FIG. 1 shows a typical design of a prior art vascular catheter wherein there is shown a heat shrink fusing sleeve 1, an outer jacket 2, a braid layer 3, a tie layer 4, a Polytetrafluoroethylene (PTFE) liner 5 and a mandrel 6.

FIG. 2 shows a catheter of this invention except the PTFE liner that is replaced by the siloxane elastomer/thermoplastic polymer 7 of this invention.

In FIG. 1, the polytetrafluoroethylene (PTFE) liner serves as the inner lubricious layer for delivery systems. PTFE is chosen for its low coefficient of friction compared to alternative polymers available in the market. PTFE can be extruded as tubing with extremely thin walls making it ideal for vascular catheter components where small size and uniform diameters are paramount. PTFE can be used to provide an exceedingly smooth inner surface.

The smooth PTFE inside diameter (ID) of these catheters reduces friction against various catheter technologies such as stents, balloons, or atherectomy devices as they are pushed through the tight confines of the catheter lumen. If the catheter ID is not of sufficient lubricity, devices such as stents can collapse in an accordion-like manner as they are being pushed through the catheter lumen. The effect of increased lubricity of the catheter ID is a reduced deployment force of catheter devices as they are passed through the lumen increasing the likelihood of a successful procedure.

PTFE, as a molecule, has features starting with the molecular structure, specifically that it orients in a helical structure. This kind of molecular packing allows for a tightly packed structure that is still very open on the atomic level due to carbon fluorine bonds. This kind of packing leads to low friction and excellent physical properties.

Despite PTFE being a great chemistry, over the last several years, PTFE chemistry has been grouped under the umbrella of Per and polyfluoroalkyl substances (PFAS) which has come into question and now has been widely labeled as “forever chemicals” due to the fact that they are widely present in the environment, accumulate in humans, and do not break down quickly.

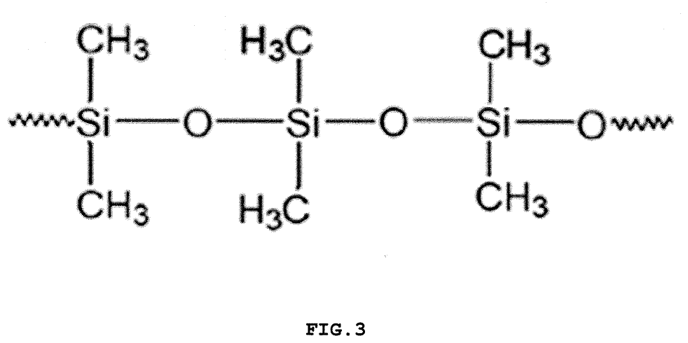

Similar to PTFE, silicone elastomer chemistry shares certain parallels when it comes to properties and molecular packing. Since “silicone” is a non-chemical name, the patentee herein defines silicone elastomer to mean some of a core structure of what is shown in FIG. 3. Additionally, there may be other functional groups or crosslinks located on the backbone of the molecule.

By leveraging properties of commonly used resins in catheters, such polyamides, polyolefins, thermoplastic polyurethane, and polyesters, it is possible to hybrid these with silicone elastomer in such a way that allows for lubricity and physical properties similar to PTFE. Within this design, a new type of catheter liner is created that closely matches the performance attributes of PTFE, however it allows for a broader range of properties.

Since PTFE is a pure molecule, its properties are based on the chemistry of PTFE. In the case of this new catheter liner design, there is an ability to vary the ratio of components, control the hybridization of the components, to be able to provide some amount of tunability on properties. Specifically, a catheter liner could be made to have higher or lower flexibility, higher or lower strength and stiffness, varying temperatures for melting and bonding, and varying levels of lubricity.

For the purpose of this polymer composition, hybridization would be defined as a simple mixture or with increased levels of bonding from hydrogen bonding to covalent bonding to ionic bonding within the species of the hybrid. Given the components of the hybrid, this allows for processing by traditional polymer processing techniques including extrusion, molding, pultrusion, dip coating, powder coating, and other coating processes.

What is claimed is:

1. A vascular catheter liner extruded from a silicone elastomer hybridized with a thermoplastic polymer selected from the group consisting of:
i) polyamides;
ii) polyolefins;
iii) polyurethanes;
iv) polyesters;
v) styrenic resins, and blends of any of i), ii), iii), iv), and v).

2. A silicone elastomer hybridized with a thermoplastic polymer selected from the group consisting of:
i) polyamides;
ii) polyolefins;
iii) polyurethanes;
iv) polyesters;
v) styrenic resins, and, blends of any of i), ii), iii), iv), and v).

3. A silicone elastomer as claimed in claim 2 wherein the hybridization is a simple blend of the silicone elastomer and at least one thermoplastic polymer.

4. A silicone elastomer as claimed in claim 2 hybridized with a thermoplastic polymer from the family of polyamide, polyolefin, polyurethane, polyester, or styrenic resins, or blends thereof, extruded into a catheter liner where the hybridization is based on increasing the hydrogen bonding between the components.

5. A silicone elastomer as claimed in claim 2 hybridized with a thermoplastic polymer from the family of polyamide, polyolefin, polyurethane, polyester, or styrenic resins, or blends thereof, extruded into a catheter liner where the hybridization is based on covalent bonding and between the components.

6. A silicone elastomer as claimed in claim 2 hybridized with a thermoplastic polymer from the family of polyamide, polyolefin, polyurethane, polyester, or styrenic resins, or blends thereof, extruded into a catheter liner where the hybridization is based on covalent bonding and hydrogen bonding between the components.

7. A silicone elastomer as claimed in claim 2 hybridized with a thermoplastic polymer from the family of polyamide, polyolefin, polyurethane, polyester, or styrenic resins, or blends thereof, extruded into a catheter liner where the hybridization is based on ionic bonding between the components.

8. A silicone elastomer as claimed in claim 2 hybridized with a thermoplastic polymer from the family of polyamide, polyolefin, polyurethane, polyester, or styrenic resins, or blends thereof, extruded into a catheter liner where the hybridization is based on ionic bonding and covalent between the components.

9. A silicone elastomer as claimed in claim 2 hybridized with a thermoplastic polymer from the family of polyamide, polyolefin, polyurethane, polyester, or styrenic resins, or blends thereof, extruded into a catheter liner where the hybridization is based on ionic bonding and hydrogen between the components.

10. A silicone elastomer as claimed in claim 2 hybridized with a thermoplastic polymer from the family of polyamide, polyolefin, polyurethane, polyester, or styrenic resins, or blends thereof, extruded into a catheter liner where the hybridization is based on ionic bonding, covalent bonding, and hydrogen the between components.

11. The vascular catheter liner as claimed in claim 1 further comprising one or multiple functional identifications additives incorporated into the hybridized polymer.

12. The silicone elastomer as claimed in claim 2 further comprising one or multiple functional identifications additives incorporated into the hybridized polymer.
